(19) United States
(12) Patent Application Publication
(10) Pub. No.: US 2019/0015624 A1
(43) Pub. Date: Jan. 17, 2019

(54) INFLATABLE FLOAT TANK

(71) Applicant: ZEN FLOAT CO. LLC, MURRAY, UT (US)

(72) Inventors: WILLIAM CHARLES HILL, MACCLESFIELD (GB); SHANE JOSEPH STOTT, SALT LAKE CITY, UT (US); SEAN GALE STOTT, COTTONWOOD HEIGHTS, UT (US)

(73) Assignee: ZEN FLOAT CO. LLC, MURRAY, UT (US)

(21) Appl. No.: 16/135,741

(22) Filed: Sep. 19, 2018

Related U.S. Application Data
(60) Provisional application No. 62/560,361, filed on Sep. 19, 2017.

(51) Int. Cl. A61M 21/00; A61H 33/00; A61M 21/02; E04H 15/12; E04H 15/34; E04H 4/00; E04H 4/10

(52) U.S. Cl. CPC A61M 21/0094; A61H 33/6005; A61M 21/02; E04H 15/12; E04H 15/34; A61H 2033/048; E04H 4/108; A61H 2201/10; A61H 2201/0228; A61H 2201/0161; A61H 2201/0157; E04H 4/0056

(57) ABSTRACT

An inflatable float tank for providing a therapeutic sensory deprivation session. The inflatable float tank includes opaque inflatable sidewalls including a drop-stitch material. The drop stitch material includes opposing walls with stitched threads extending at acute angles from one another. When uninflated these internal stitch structures are allowed to collapse and provide substantially foldable and pliable deflated sidewalls as opposed to substantially rigid sidewalls upon inflation. The inflatable float tank is filled with float solution including water and Epsom salt heated to skin temperature so as to provide a highly buoyant float environment with a lack of sensory stimulation. The inflatable float tank exhibiting improved insulation within the tank and ease of assembly. The inflatable float tank and assembly thereof doesn't require rigid support structures such as poles and joints. And, the inflatable float tank is substantially foldable in an uninflated state and is substantially rigid in an inflated state.

### CROSS-REFERENCE TO RELATED APPLICATIONS

This application claims the benefit of and priority to U.S. Provisional Patent Application 62/560,361 filed Sep. 19, 2017 entitled INFLATABLE FLOAT TANK, the contents of which are incorporated herein by reference in its entirety and for all purposes.

This application shares common inventors with U.S. patent application Ser. 14/035,494 filed Sep. 24, 2013 entitled FLOAT TENT, now U.S. Pat. No. 9,345,852 issued May 24, 2016; and U.S. patent application Ser. No. 15/162,009 filed May 23, 2016 entitled FLOAT TENT, now U.S. Pat. No. 9,669,184 issued Jun. 6, 2017.

### BACKGROUND

Floatation therapy is a medical treatment using sensory deprivation within a floatation tank, floatation tent, or float room. Years of well-documented tests show that floatation tank therapy not only has an immediate effect on pain relief and elimination of stress, it also promotes ‘whole-brain’ thinking. Many athletes, writers, and artists have experienced enormous benefits from floatation therapy utilizing floatation tanks.

Floatation therapy, generally entails spending 45 to 60 minutes in a small, pitch-black, soundproof room floating naked in a pool of skin-temperature water that is fully saturated with Epsom salts causing a subject to simply float, completely devoid of any outside stimulus. Floatation therapy is also called a sensory deprivation experience. Some facilities use a floatation pod or floatation tank to allow the body to feel suspended in space. Scientifically known as magnesium sulfate, Epsom salt baths have been used for generations for relaxation and muscle soreness. Relaxation benefits are more likely to come from the combined impact of the water and weightlessness. The salt changes the quality of the water, making it feel silky and slippery. Warmth is a known muscle relaxant, and being free from gravity helps your muscles fully relax.

Recent meta-analysis of 27 individual studies including nearly 450 participants found that such restricted environmental stimulation therapy (REST) lowered levels of the stress hormone cortisol, lowered blood pressure, and improved sense of well-being. In one 2014 study of 65 people suffering from stress and/or muscle pain, those who underwent 12 float sessions enjoyed significant decreases in stress, depression, anxiety and reported less pain and increased optimism and improved sleep quality compared to those who received no float therapy. See “Beneficial effects of treatment with sensory isolation in flotation-tank as a preventive health-care intervention—a randomized controlled pilot trial” BMC Complement Altern Med. 2014; 14: 417, Oct. 25, 2014. In fact, many athletes such as Tom Brady have touted the use of floatation therapy. It has been reported that Tom Brady, who is 41 years old and plays professional football in the NFL to this day, has a float tank in his house. However, a float tank often too expensive for the average consumer, or floatation therapy subject, even in instances of serious physical and/or mental need. And, installation of large traditional float tanks or flotation pods may be structurally or physically impossible to erect or install within a person's home. Moreover, the time consuming assembly of prior float tanks or float tents made by the common inventors to this patent application may be daunting to the average or unskilled consumer.

The inventors of this patent application are also inventors of issued U.S. Pat. Nos. 9,345,852 and 9,669,184 directed to the FLOAT TENT as well as several international patents. The float tent has helped hundreds, if not thousands, of participants realize the benefits of REST using their Float Tent technology for in-home floatation therapy. As discovered by the inventors, and disclosed herein, there remains a need in the art for improved float tanks, related floatation methods, and improved float tank-like systems.

### BRIEF SUMMARY OF THE INVENTION

This Summary is provided to introduce a selection of concepts in a simplified form that are further described below in the Detailed Description. This Summary is not intended to identify key features or essential characteristics of the claimed subject matter, nor is it intended to be used as an aid in determining the scope of the claimed subject matter.

A therapeutic inflatable float tank is disclosed for providing a sensory deprivation state for a subject in order to inhibit light, temperature, and physical stimulus to the subject while floating in the therapeutic inflatable float tank. The inflatable float tank includes opaque light inhibiting inflatable walls, the walls including an internal drop-stitch support structure that lends rigidity to the walls when inflated but is substantially collapsible, flexible, and foldable when uninflated. The inflatable float tank includes a heater configured to maintain a temperature of a float solution between 88 and 99 degrees Fahrenheit so as to inhibit temperature stimulus to the user. The inflatable float tank includes a base connected to the inflatable walls so as to provide a water-tight connection between the base and walls, the inflatable sides and base dimensioned to hold less than 24 inches deep of float solution and dimensioned sufficient for a subject to float in the float solution without touching the pool sides or the base of the pool.

The walls provide thermal insulation to the interior of the therapeutic inflatable float tank due to a substantially air-filled void between the drop-stitch connected inner surfaces of the walls. The inflatable float tank can be devoid of solid support structures such as poles, joints, and other rigid structural supports. The inflatable float tank can be devoid of metal or plastic internal or external support structures.

The base can be inflatable for providing thermal insulation between a floor and the interior of the inflatable float tank. The heater can be a radiant heater for substantially silent heating of the float solution during a float session. The base can have an internal drop-stitch support structure that lends rigidity to the base when inflated but is substantially collapsible, flexible, and foldable when uninflated.

The one or more walls can have an opening for the subject to enter the therapeutic inflatable float tank. The inflatable float tank can include an inflatable pivotable door made having an internal drop-stitch support structure that lends rigidity to the door when inflated but is substantially flexible, collapsible, and foldable when uninflated. On or more walls can be seamless between opposing ends of the at least two walls. One or more walls can be triangular and curved in shape without planar surfaces so as to inhibit internal condensation drip therefrom. The walls can be devoid of seams and undulations.

The walls can be inflated to a pressure of at least 15 or at least 20 psi resulting in substantial rigidity of the walls as compared to the uninflated state of the float tank. The float solution can include water having at least 3% salinity of Epsom salt dissolved therein. For example, the float solution can include water having at least 800 pounds of Epsom salt dissolved in less than 14 inches of water held within the inflatable float tank resulting in at least 3% salinity of Epsom salt in the water. In some embodiments, the water may be about 10 to 12 inches deep and defined by the interior walls being about 8 feet long and 4 feet wide and held at about 91-95 degrees Fahrenheit. Thus, according to certain preferred embodiments the volume of water the float tank is designed to hold can be between about 26 and 32 cubic feet of float solution i.e. about 200 to 240 gallons of float solution. However, according to other designs disclosed herein the inflatable float tank can hold between 160 gallons to 480 gallons of float solution, however.

A method for assembling a float tank can include inflating the float tank to a rigid upright position, filling the inflatable float tank with between 8 and 14 inches of the float solution, and heating the float solution to between 91 and 99 degrees Fahrenheit so as to provide a sensory deprivation float therapy session.

A kit for floatation therapy can include an inflatable float tank, a heater, a circulation pump, a filtration system, and 800 pounds of Epsom salt. The kit can be devoid of poles and rigid supports.

A method for manufacturing an inflatable float tank can include selecting a wall thickness for side walls of the inflatable float tank, the wall thickness being selected to provide thermal insulation and sidewall rigidity when inflated, and manufacturing the inflatable float tank comprising a drop-stitch material, the drop stitch material includes opposing side walls with stitched threads extending at acute angles from one another, where when uninflated these internal stitch structures are allowed to collapse and provide a substantially foldable and pliable deflated float tank as opposed to substantially rigid sidewalls upon inflation.

An inflatable float tank can include opaque inflatable sidewalls including a drop-stitch material, the drop stitch material includes opposing walls with stitched threads extending at acute angles from one another, where when uninflated these internal stitch structures are allowed to collapse and provide a substantially foldable and pliable deflated sidewalls as opposed to substantially rigid sidewalls upon inflation, the inflatable sidewalls being curved and devoid of seams and devoid of planar surfaces so as to inhibit condensation dripping therefrom. The inflatable float tank can include an inflatable base including a drop-stitch material, the drop stitch material includes opposing walls of the base with internal stitched threads extending at acute angles from one another, where when uninflated these internal stitch structures are allowed to collapse and provide a substantially foldable and pliable deflated base as opposed to the substantially rigid base upon inflation;. The inflatable float tank can include an opening in a wall for a subject to enter the interior of the inflatable float tank. The inflatable float tank can include float solution filing between 8 and 12 inches of the inflatable float tank, the float solution comprising water and at least 800 pounds of Epsom salt dissolved in the water. The inflatable float tank can include a radiant heater for heating the float solution to skin temperature so as to inhibit sensory stimulation during the float session.

Additional features and advantages of the invention will be set forth in the description which follows, and in part will be obvious from the description, or may be learned by the practice of the invention. The features and advantages of the invention may be realized and obtained by means of the instruments and combinations particularly pointed out in the appended claims. These and other features of the present invention will become more fully apparent from the following description and appended claims, or may be learned by the practice of the invention as set forth hereinafter.

### BRIEF DESCRIPTION OF THE DRAWINGS

To further clarify the above and other advantages and features of the present invention, a more particular description of the invention will be rendered by reference to specific embodiments thereof which are illustrated in the appended drawings. It is appreciated that these drawings depict only typical embodiments of the invention and are therefore not to be considered limiting of its scope or necessarily drawn to scale. The invention will be described and explained with additional specificity and detail through the use of the accompanying drawings in which:

FIG. 1 illustrates a front perspective view of an inflatable float tank;

FIG. 2 illustrates a left view of an inflatable float tank;

FIG. 3 illustrates a front perspective view of an inflatable float tank showing access to the interior via a door and opening;

FIG. 4 illustrates the internal support structure of the walls and base of an inflatable float tank;

FIG. 5 illustrates a front, side, and rear view of an inflatable float tank describing various examples of potential and preferred dimensions of the inflatable float tank;

FIG. 6 illustrates a side cross-sectional view of a subject during a float session in an inflatable float tank;

FIG. 7 illustrates a cross section view of a subject during a float session along with various examples of features, equipment, and components of the inflatable float system;

FIG. 8 illustrates a top view of a float tank according to an example embodiment;

FIG. 9 illustrates a front perspective view of a float tank;

FIGS. 10A-10F illustrate a method of assembling the float tank including inflating the float tank from a collapsed state to a ridged inflated state;

FIG. 11 illustrates other design and shape configurations according to other embodiments of an inflatable float tank in addition to the preferred embodiments shown and described in FIGS. 1-9;

FIG. 12 illustrates a method for manufacturing and assembling an inflatable float tank as well as providing a kit for deployment of a float tank; and

FIG. 13 illustrates a method for assembling an inflatable therapeutic float tank or inflatable float tank kit.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the figures, can be arranged, substituted, combined, separated, and designed in a wide variety of different configurations, or in part, all of which are explicitly contemplated herein.

The present invention includes an inflatable float tank exhibiting many improvements over the prior art. Such improvements can include improved portability, improved thermal insulation, decreased time in setup, improved strength, improved sensory damping, lower likelihood of condensation drip, and decrease in assembleable parts.

The improvements in portability, setup time, and reduced part assembly results in a simplified experience for the user. In fact, such assembly may be accomplished in as few as 5-10 minutes in some instances. For example, such inflatable float tank assembly can be accomplished by simply inflating the float tank into its internally structurally supported and designed shape. The shape to which the float tank is inflated becomes substantially rigid, hard, and strong due to the internal material drop-stich structure from which it is manufactured. In fact, according to some preferred embodiments, after placement in a desired location, assembly of the float tank is accomplished using a simple air inflation pump such as a hand, foot, or electrical air pump so as to erect the float tank. Subsequent use only requires filling the float tank with water to the desired water level, adding Epsom salt to achieve the desired high level of salinity, and connecting the heater and filtration systems to the float tank along with electrical power. Disassembly may be as easy as disconnecting the heater and filtration systems, draining the salinated water, and deflating the float tank so as to move, store, or ship the float tank to another location.

Further important improvements particular to float tank technology include increased thermal insulation of the walls and floor of the float tank for retaining the heated properties of the floatation solution retained therein. Such increased insulation of the interior chamber from the ambient outside conditions includes decreased heat transfer through the walls and floor of the float tank resulting in greater than 40% improvement in heat retention therein as compared to single-wall designs. In addition, the floor of the float tank may be made of the same or similar drop-stitch construction as the sidewalls forming a continuous 360 degree insulation chamber within the float tank so as to an entirely thermally encapsulated chamber. For example, in instances where the float tank is disposed upon a concrete floor there is a deep capacity for absorbing heat conducted through the floor of a float chamber. According to the teachings herein, use of a relatively thick drop-stitched base material for the float tank further retains a high amount of heat within the float tank as opposed to conduction through a single walled floor material such as rubber or plastic.

Additional improvements include the elimination of seams, seals, cracks, and crevices that attract biological growth and mineral accumulation. Moreover, a smooth continuous integrated surface is less likely to result in condensation drip, which can be distracting for a float subject and overall ruin the float experience. In fact, as shown in the drawings and discussed herein, the sidewalls can be substantially devoid of seams, irregular surfaces, and undulations. The sidewalls can be relatively steep, smooth, curved or straight so as to avoid abrupt changes in direction often resulting in condensation drip. For example, the internal walls and external walls can have a consistent thickness and smooth continuous surface as a result. This smooth continuous surface is provided so as to create a consistent float experience with avoidance of condensation accumulation, accumulation of biological material, or accumulation of mineral deposits.

The walls of the inflatable float tank have the unique integrated internal support structures eliminating internal or external support structures. The walls transform from a flexible collapsed state to a substantially rigid inflated state by simple inflation. The substantially rigid inflated state is vertically and horizontally rigid using the internal support structure of the drop-stitch material. In fact, the inventor of this patent application have placed over 200 pounds upon, and hanging from, the peak of the float tank and noticed very little deformation thereto under such structural weight while using no reinforcing rigid solid support structures.

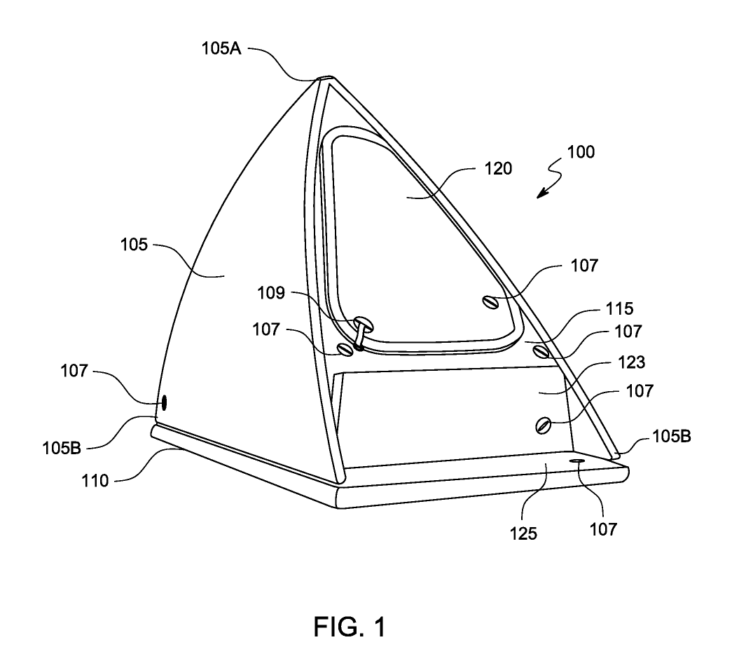

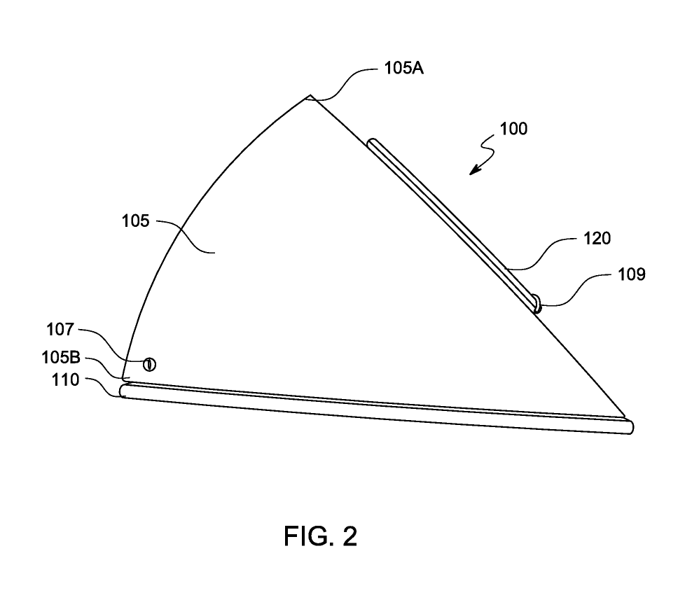

Referring to FIGS. 1-3, the inflatable float tank 100 can include sidewalls 105 that are integrated inflatable walls 105 without a seam from the top 105A of the sidewalls 105 to the bottom 105B of the sidewalls 105. The integrated inflatable sidewalls 105 being a single inflated structure with opposing inner and outer walls connected together by drop-stitch fibers providing less parts, less (or no) seams, and simplified assembly. The reduction of seams of the float tank 100 sidewalls 105 can eliminate ledges, cracks, overlaps, and other areas prone to biological growth and mineral accumulation.

The float tank 100 can further have increased height and increased vertical slope in sidewall 105 construction so as to decrease the likelihood of accumulation of condensation and condensation dripping from the interior of sidewalls 105. As previously discussed, the sidewalls 105 reduce heat transfer there through as the walls 105 mostly contain air which is a relatively poor heat conductor, the conduction through the individual drop stitch fibers being substantially negligible.

Because of the decreased heat transfer through the sidewalls 105 of the float tank 100 the interior surface of the float tank 100 sidewalls 105 stays relatively warmer as compared to the interior surface of a single layer sidewall according to previous designs. Thus, the exterior surface of the side walls 105 may be closer to room temperature temperature of the lower ambient temperature outside the float tank 100 while the interior surface of the sidewalls 105 remains closer to the higher interior temperature of the heated float solution disposed in the interior of the float tank 105. As a result, condensation is less likely to accumulate on the interior surface of the sidewalls 105 of the float tank 100 because condensation tends to collect as droplets on a cold surface, as opposed to collecting on a relatively consistently warmer interior surface of the sidewalls 105 of the float tank 100.

As previously discussed, the assembly and disassembly of the float tank 100 is greatly simplified as the float tank 100 is devoid of rigid plastic or metallic support structures requiring assembly and dis-assembly. However, when inflated the drop-stitch structure of the float tank 100 sidewalls 105 and base 110 become substantially rigid in a predetermined shape to which the components of the float tank 100 are manufactured. The sidewalls 105 are self-supporting when inflated due to the manufactured internal drop-stitch construction. This substantially rigid and strong inflated sidewall 105, inflated front wall 115, inflated rear wall 122, inflated door 120, and inflated base 110 construction is realized by the internal drop-stich material used to construct the inflatable float tank 100. Using the matrix of V-directional internal drop-stitch threads, the sidewalls 105, front wall 115 rear wall 122, base 110 and door 115 become substantially rigid. Sidewalls 105 can be angled, curved, pyramid, and triangular-shaped in some designs so as to provide a smooth and continuous interior surface resistant to condensation drip. The walls 105, 115 and 133 are smooth and continuous avoiding corrugation, unevenness, seams, and other changes in surface characteristics. The rigid and inflatable base 110, opening 130, and entryway provided by vertical wall 123, rigid and inflatable platform 125, and door 120 are provided for easy access to the interior of the float tank 100. Such rigid sidewall 105 construction provided when inflated has been found by the inventors to provide sufficient structural rigidity to support over 200 pounds when placed directly on the top of the float tank 100 without substantial deformation or collapse thereof.

The drop-stitch material is disposed between opposite interior surfaces of each sidewall 105 lined with a light and/or water impervious material, such as a polymer layer. The drop-stitched material includes spaced threads that when held under tension provide rigidity to the opposing sidewalls 100 due to the tensile force applied by such threads to the internal surfaces of the sidewalls 105. Such threads can be disposed at angles such as in the instance of V-drop stitching where threads are disposed at differing tensile angles to the opposing interior surface resulting in tensile vectors of force in various angles thereto thereby creating the rigidity under internal air pressure.

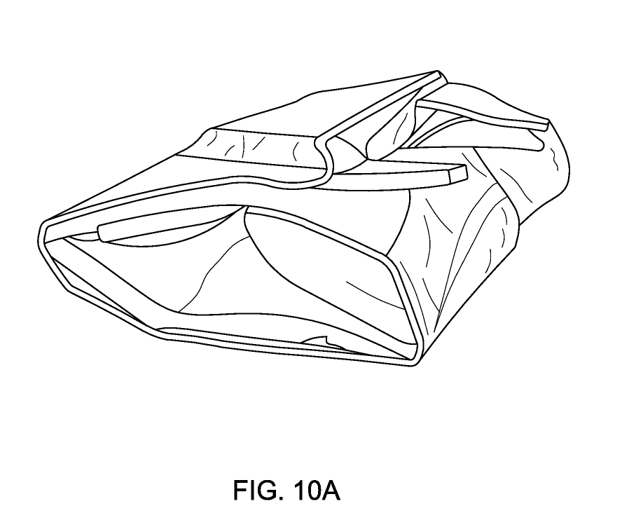

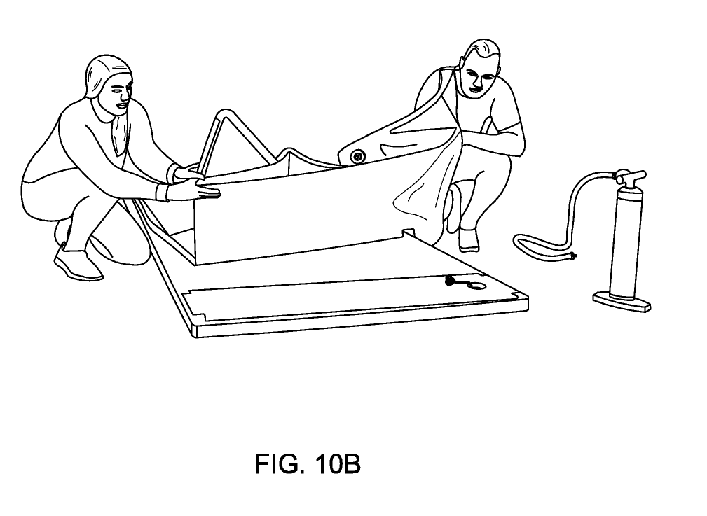

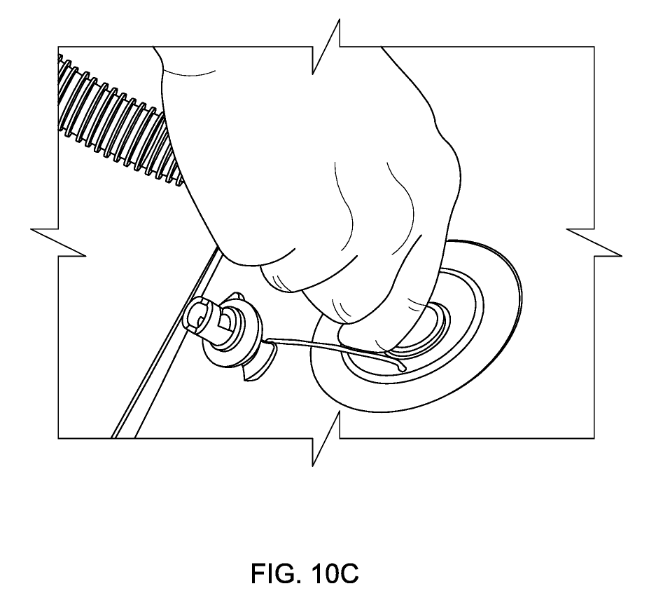

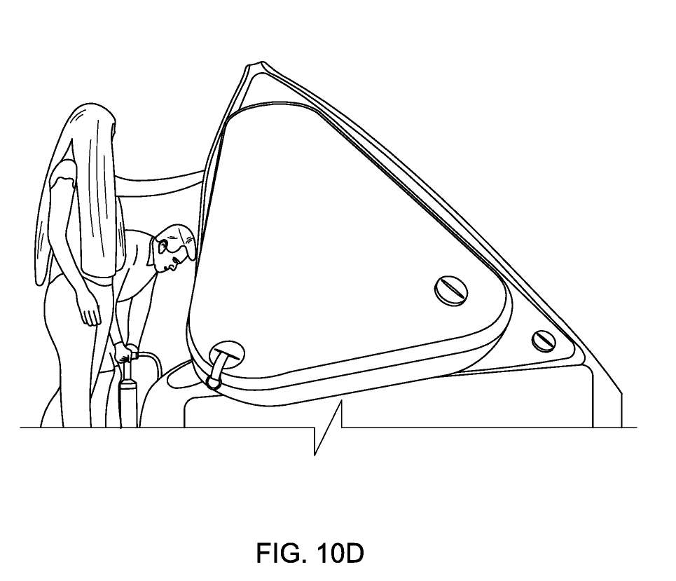

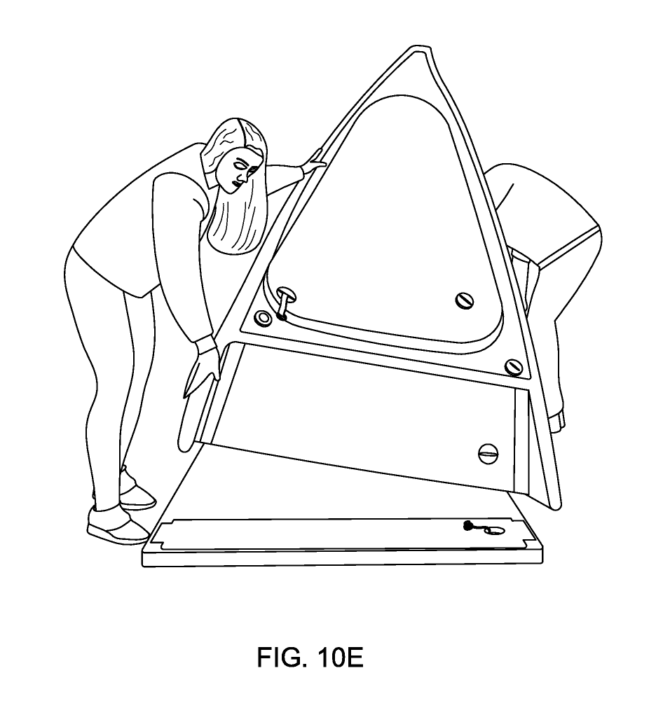

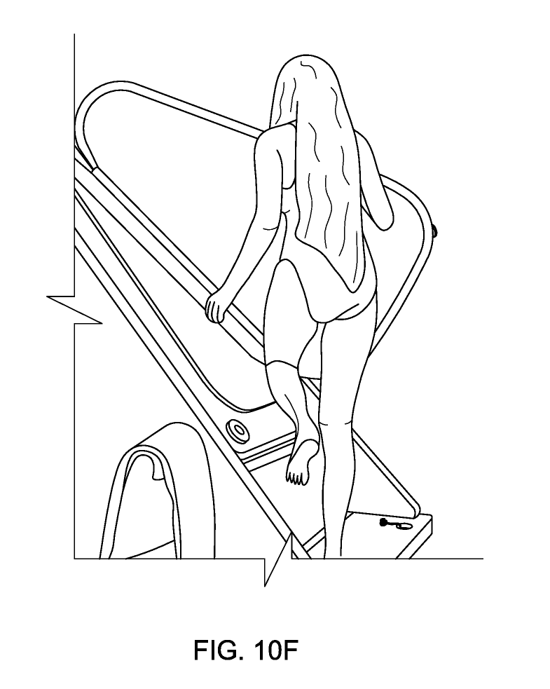

According to the embodiments shown in FIGS. 1-3 and 5-9 two sidewalls 105, 305 are substantially triangular, curved or straight, and extend entirely from the upper tip 105A, 305A of the float tank 100, 300 to the bottom 105B, 305B of the float tank 100, 300 where a base 110, 310 is formed. The shape of the float tank 100, 300 may take other forms as discussed below with reference to FIG. 10. Referring still to FIGS. 1-3, the front wall 115 of the float tank 100 includes a vertical entryway wall 123. The entryway wall 123 meets a recessed step or platform 125 disposed upon a surface of the base 110 for providing improved access to the interior of the float tank 100. The door 120 of the float tank 100 is made of a similarly inflatable and rigid drop-stitch material that is substantially rigid when inflated yet collapsible and foldable when uninflated.

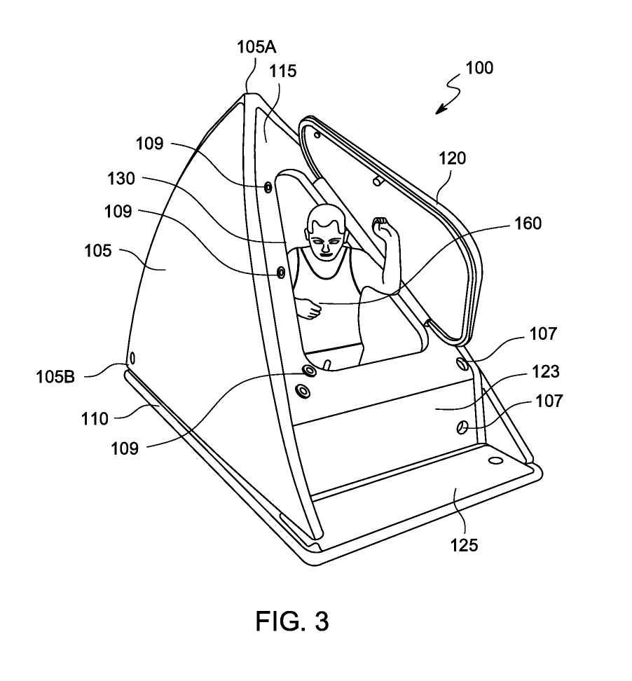

Similar to the sidewalls 105, the base 110 provides a high level of insulation between the interior of the float tank 100 and the ground as opposed to a single layer plastic or rubber base as in the prior designs. The door 120 is pivotably held over the opening of the front wall 115 of the float tank 100 and may be opened to enter the float tank 100 as shown in FIG. 3. The front wall 115 and door 120 may be include fixtures, grommets, buttons or other fasteners 109 and sealing features for holding the door securely and releasably against the front wall 115 of the inflatable float tank 100. The fixtures 109 may be accessible from inside the float tank 100 so as to release the door 120 and exit the float tank yet securely fast then door 120 to the front wall 115 to retain heat therein and suppress noise, light and cold air from entering the interior of the float tank 100.

The float tank 100 is capable of holding between 8 and 24 inches of float solution including water and Epsom salt that can be used in the floatation therapy. In some preferred embodiments, the float tank 100 is designed to hold between 8 and 14 inches of float solution with at least 3% salinity. The inflatable float tank 100 can include the inflatable walls 105 and 120 as well as front wall 115, entryway wall 123, and rear wall 122. The float tank 100 filled with heated salinated water provides improved suppression of physical stimulus in the form of heat transfer from the skin of the subject for a long duration of time in addition to the suppression of light and sound. In fact, it has been found that the use of the drop-stitched walls also provides improved suppression of sound and light from entering the interior of the float tank 100 as opposed to the single wall design of the prior designs in addition to the increased thermal insulation.

Thus, the inflatable float tank 100 can also be stored and shipped in a deflated state without requiring assembly or disassembly of a support structure having rigid or semi-rigid supports. The deflated float tank 100 can be packaged and shipped in the deflated state. Upon receipt the float tank 100 may be removed from any packaging and inflated to a sufficient internal wall pressure to provide the shape and upright structure of the inflatable float tank 100. For disassembly and subsequent transport, the float tank 100 may be drained of the float solution and deflated for transport and use at another location. Once placed at a different location for use the float tank 100 may be reinflated to its substantially rigid predetermined erect form and filled to the desired level of heated float solution.

The inflatable float tank 100 can be configured as a waterproof inflatable structure designed for a subject to lie down in and float within such structure without touching the walls 105, 115, 122, or base 110. In some embodiments, the float solution can be filled to between about 8 and 24 inches in height to allow the subject to float without touching the sides 105, 120, 122 or base 110. More preferably, the float tank 100 can be dimensioned to contain salt water at a depth of between about 8 inches to about 12 inches. According to one design the float tank 100 is provided with about 800 pounds of Epsom salt, dimensioned to be four feet wide (W1), eight feet long (L1), and preferably holds about 10 inches of water thereby retaining a four foot, by 8 eight foot, by 10 inches deep float solution with at least 3 percent salinity of the Epsom salt. The inflatable float tank 100 can be waterproof and watertight so as to retain the float solution therein without leakage.

The sidewalls 105, front wall 115, vertical wall 123, entryway wall 123, base 110 and door 120 can each have an inflation port 107 for inflating each section. However, each wall and/or section may be in fluidic communication with another wall/section such that a single inflation port 107 may be used to inflate multiple portions of the inflatable float tank 100. For example, the base 110 and/or any wall 105, 115, 123, or door 120 may be coupled for inflation from a single inflation port 107. The walls of the float tank 100 may include one or more ports for accessing, circulating and filtering the float solution.

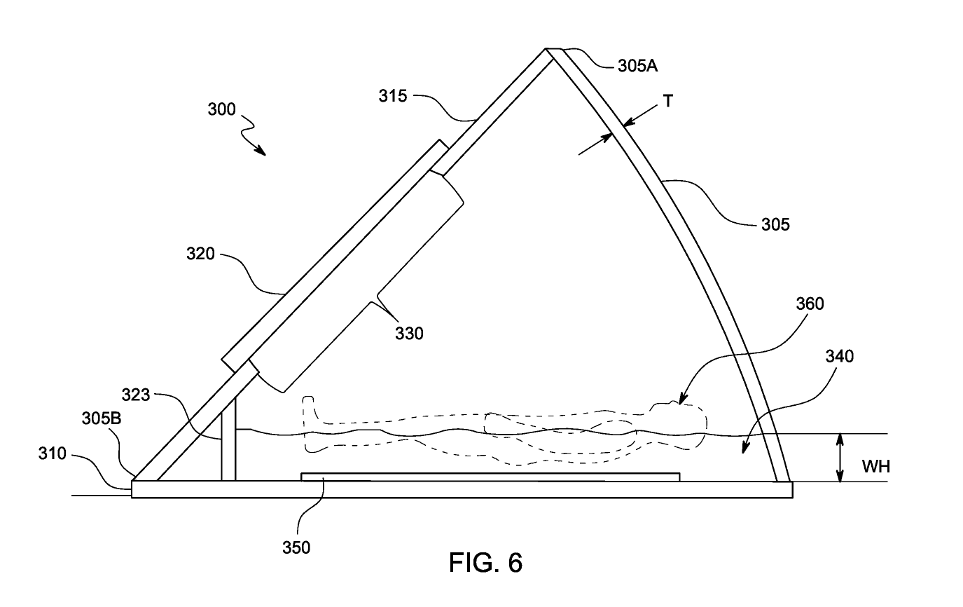

As shown in FIG. 3, the subject 160 can enter the inflatable float tank 100 via an opening 130 in the inflated front wall 115, and then lie down in the float solution (e.g. see 340 in FIG. 6) having an appropriate salt content so that the subject floats without aid or swimming action. That is, the subject can lie in the float solution and float without movement and without touching the sides or bottom of the float tank 100. In an example, the float solution can include a mixture of about 800 pounds of Epsom salt and 10 inches deep of water. The water can be heated to an exterior skin body temperature, for example between 91 and 99 degrees Fahrenheit with a heater. While a radiant heater is shown, a circulated heater can be used according to other embodiments. In one embodiment the temperature can be adjusted between 91 and 95 degrees Fahrenheit and a salinity of at least 3% Epsom salt. This float solution in the form of salt water can suspend the subject at the top level of the water creating a near zero gravity environment by effortlessly floating and maintaining the resting body at the top of the water.

The subject can have a complete lack of sensations while floating, which lack of sensations can improve the environment in the inflatable float tank 100 to be devoid of: gravity sensations, light, sound, and any feeling of temperature transfer from the body, as well as other sensations. The experience in the inflatable float tank 100 can be sufficient to remove sensations so that the mind can relax to a state of meditation or clearness. The subject can spend various amounts of time within the inflatable float tank, which can be measured or set with a timer device, such that the time can vary from 30 minutes to a few hours. The time of floating in the inflatable float tank can be sufficient to allow the subject to feel disconnected from the outside world, and to aid such a disconnectedness and relaxation state. All electronic devices or outside environmental distractors can be removed or turned off. In one aspect, the inflatable float tank can be used in a room that is isolated without electronic components that can be distracting. For example, the subject's mobile phone can be left outside of the room to enhance the disconnectedness and relaxation state.

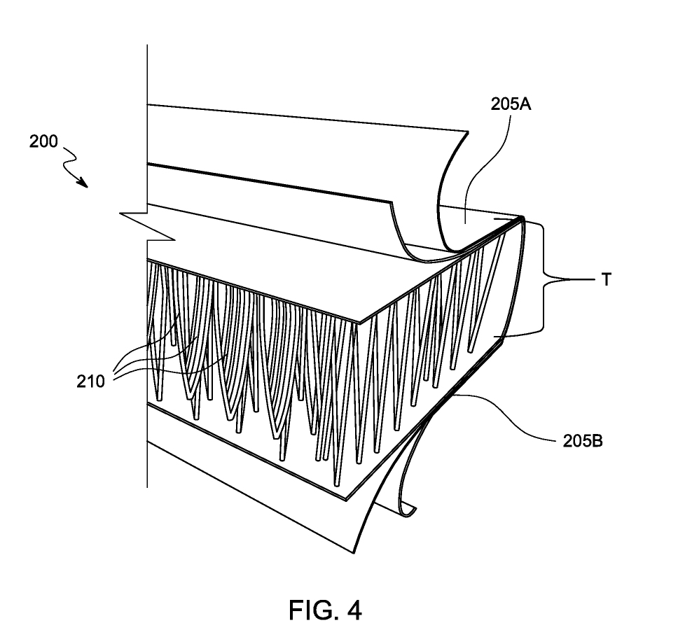

Referring to FIG. 4, the drop-stitch material 200 is shown in better detail for forming the walls 105, 115, 120, 123, base 110, and/or door 120 of the float tank 100, 300. The drop stitch material 200 is a double wall constructed fabric 205 where each wall is attached by spaced threads 210. This double wall design not only creates a rigid wall 205 when inflated but will also adds insulation value to the interior of the inflatable float tank 100, 300. For example, the heat transfer through the thickness T of the drop stitch material 200 from an exterior surface 205A to an interior surface 205B is greatly reduced. As such, the interior surface 205B may be relatively warmer than the exterior surface 205A. The improvements to insulation of the inflatable sidewalls 105, 115, 120, 123, base 110, and door 130 of the float tank 100 are important to the particular art disclosed herein so as to keep the float solution 140 (including water and dissolved Epsom salt) at a desired elevated temperature as compared to outside ambient temperature of the room and ambient outside temperature. This increased insulation value is substantial as compared to a single wall thickness and is particularly relevant to float therapy as the requirement of heating the float solution or any noise associated thereto for circulation may be eliminated.

For example, the insulation value of air has a large insulation value as compared to the heat conduction insulation value of a single polymer wall. The conduction of heat through the threads 210 of drop stich material 200 is substantially negligible as compared to the conductive value through a single polymer wall. In the art of float treatments this also enables a reduction in the circulation of float solution in such embodiment where a radiant heater is not implemented. Such improvements can result in retention of float solution at such elevated temperature during even more extended use of the float tank 100, 300 without requiring reheating or circulation of the float solution during such float sessions. And, with use of a radiant heater the float solution may be heated during a float session without undue noise created by water circulation,

The spaced threads 210 can be a V-drop stitch configuration where opposing side walls 205A and 205B can include base cloth that has stitched threads or internal structural support in distinct V-shaped directions. The thickness T can be between 1 and 5 inches. In some embodiments the wall, door, and base can be about two or three inches thick T. That is, the internal support threads 210 of the drop stitch internal side walls 205A and 205B can extend at an acute angle from one another such that the multi directional internal sidewall supports provided by the drop stitch threads 210 extend at acute angles to the internal sidewall surfaces 205A and 205B of the walls, door, and base of a float tank when inflated. When uninflated these internal stitch structures 210 are allowed to collapse and provide a substantially foldable and pliable deflated float tank as opposed to the substantially rigid sidewalls, door and base upon inflation.

The V-drop stitch technique can be used according to the present invention so as to manufacture an inflatable float tank which can be substantially collapsible and transportable when uninflated yet substantially rigid and capable of supporting the float solution (salinized water) for float sessions. A drop-stitched PVC fabric includes two walls and is therefore termed a “double wall fabric.” It typically includes opposing and parallel PVC panels connected with thin and dense columns of tens of thousands of interwoven nylon stitches holding the panels in their opposing relationship. The stitching pattern can be varied so as to provide differing degrees of rigidity, but any degree of rigidity using any known pattern requires the introduction of high pressure into the closed volume defined by the panels. Pressures over 30 psi are possible, and pressures of 15-25 psi are common, but more importantly, they are needed to provide the walls of the float tank with any meaningful degree of rigidity, particularly when the float tank is supporting the weight of the float solution and potentially the weight of a subject climbing into and out of the inflated float tank.

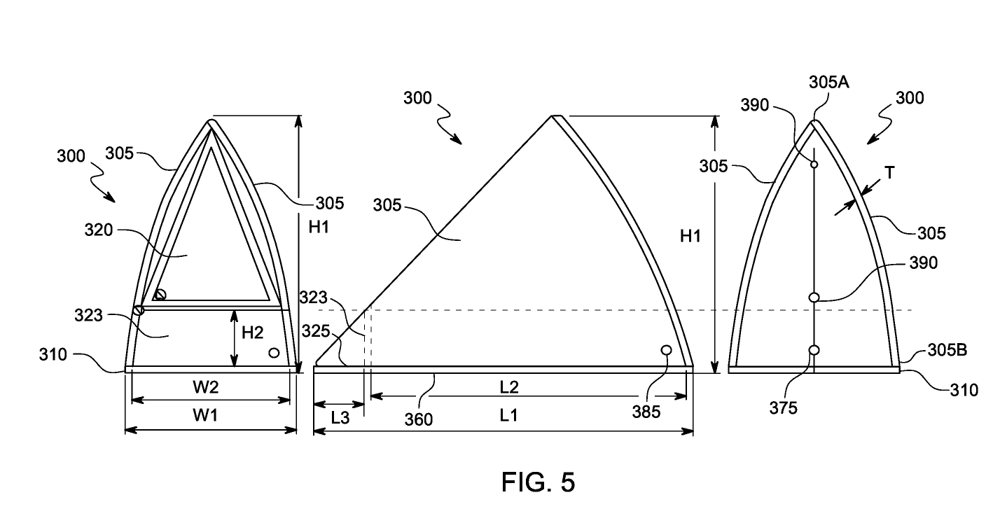

Referring to FIG. 5, various examples of dimensions of a float tank are illustrated. The float tank may have a height H1 of approximately 5 to 7 feet, a width W1 of approximately 3 to 5 feet, and a length L1 of approximately 7 to 10 feet when inflated. According to one embodiment, the height H1 is approximately 1900 millimeters, the width W1 is approximately 1300 millimeters, the width W2 is approximately 1200 millimeters, the length L1 is approximately 2880 millimeters. The sidewalls 305 may be between 1 and 5 inches thick where in FIG. 5 the width W2 may be about 1200 millimeters, L2 may be about 2400 millimeters, the height H2 can be about 420 millimeters, and length L3 may be about 380 millimeters, for example. According to one embodiment the wall 305 thickness is about 50-150 millimeters thick resulting in sufficient rigidity and thermal insulation properties. The entryway may be about 15 inches long L3 with a vertical sidewall of about 16.5 inches H2 in some embodiments.

FIG. 6 illustrates a cross-sectional side view of an inflatable float tank 300. The inflatable float tank 300 is filled with a float solution 340 with a water height WH, which can be between about 6 inches and 24 inches, preferably between 8 and 12 inches, with a salinity of about 3% to 3.5% dissolved Epsom salt, preferably saturated or super-saturated salinity. According to one example, the water height WH can be about 10 inches deep and has 800 pounds of dissolved Epsom salt therein creating a salinity of about 3.5% held at between about 91 degrees Fahrenheit and 95 degrees Fahrenheit, preferably about 93.5 degrees Fahrenheit for some subjects.

The size and location of the entryway including base surface 325 and entryway wall 323 to the opening 330 can be configured to accommodate such water height WH without the float solution spilling out of the float tank 300. Vertical internal distance H2 of the wall 323 to the opening 330 form the base 310 can include water height WH plus about 2 to 10 inches such that the float solution 340 is retained within the float tank 300 and the subject 360 is able to enter and exit the float tank 300 through the opening 330. The inflatable float tank 300 can have the base 310 with a width W1 that can vary depending on the size of the subject to use the inflatable float tank 300, vary on the space available for the inflatable float tank 300, or for any other reason. The size of the float tank 300 being designed for the size of the subject 360 to float with arms and legs fully extended outward or floating with the arms and legs close to the body. Some examples of width W1 of the pool base 310 can be at least about 4 feet to 6 feet, for example. The length L1 of the base 310, as per FIG. 5, can vary, such as at least about 6 feet, 7 feet, 8 feet, 9 feet, or 10 feet. The dimensions of the base 310 can be configured to accommodate a floating human, such as a child, teen, or adult whether male or female. In some embodiments the size may be increased proportionally to an increase in size or weight of the subject 360.

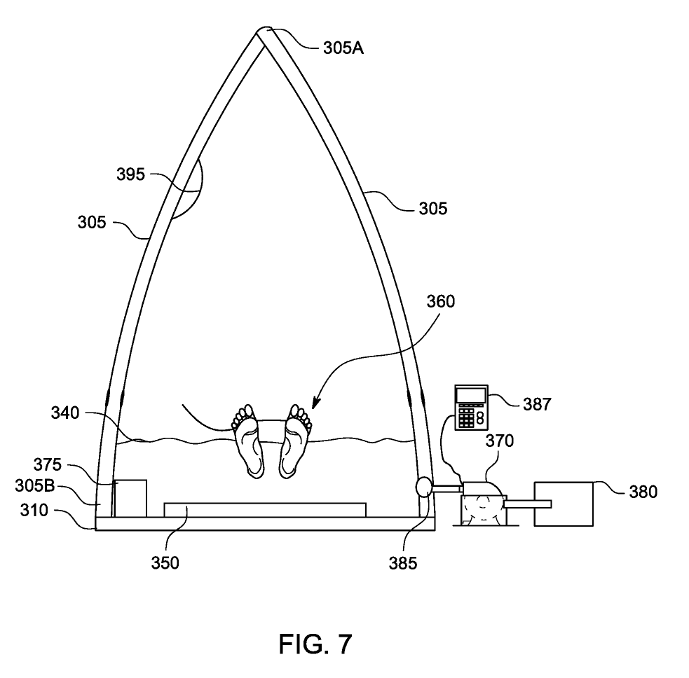

Referring to FIGS. 6 and 7 the inflatable float tank 300 can include a combination of components that provide the various functionality for use of the float tank 300. Such components may be example of components that can be used in the previously discussed embodiment of FIGS. 1-3. Other examples of components are disclosed in the patent documents previously discussed and incorporated herein by reference. Such components can include a radiant heater 350, circulation pump 370, UV/ozone device and/or filter 380, salt reservoir 375, or other component that can be at least partially submerged in the float solution 340 or may be external to the inflatable float tank 300 as shown in FIG. 7. While FIG. 7 shows the components disposed on the pool base 310 or external to the float tank 300 with access to the float solution through a port 385 in the sidewall 305, such components may be disposed on the pool wall 305 or integrated into the base 310 or sidewalls 305. For example, the radiant heater 350 is shown disposed upon the base 310 but may be part of the base 310 or disposed around a periphery of the float tank 300. Similarly, the circulation pump 370 is shown external to the float tank 300 but may be disposed within the float tank 300 or have peripheral ducting for balanced circulation of filtered float solution.

The float tank 300 can include components such as a timer (e.g., to time the floatation duration), sound device (e.g., any device that plays sound ranging from music to ambient sounds, such as waves, crickets, heartbeat, or other), light (e.g., to provide one or more lights of one or more colors or changing colors or to simulate stars or other), fan, vent 390 (e.g., to vent air, humidity, smells, or other from the inside to outside the inflatable float tank 300), or other components or devices.

In one aspect, a controller 387 can be used to control any of the components of the inflatable float tank 300, such as the heater 350 and circulation pump 370 as well as other functionality such as light and music. The controller 387 can be wired to the components, or the controller 387 and components can be configured to be wireless for wireless control of the components. In one example, the controller 387 is a handheld communication device (e.g., Droid, iPhone, tablet, etc.) with an application that functions as a controller application.

The inflatable float tank 300 may also include one or more grab handles 395 to facilitate entry into the inflatable float tank 300 or to facilitate rising from the float solution or exiting the inflatable float tank 300. Various D-rings and other fasteners may be disposed within or on the exterior of the float tank 300 for hanging additional devices or providing additional functionality.

If external filtration isn't desired then the filtration method can be the same as previously described. This includes a mechanical filter, submersible UV filter and submersible pump that is placed inside the inflatable float tank. Another property of the filtration system that can be included is how the solution from the pump is introduced back to the inflated float tank 300. This reintroduction of the float solution includes spreading orifices at least partially or entirely disposed down the length of the tank portion 320 allowing for distribution of the filtered float solution throughout the pool portion 320 of the inflated and filled float tank 300, which helps circulate and aerate the heated float solution and provides a cleaner solution throughout the pool portion 320.

In one embodiment, the inflatable float tank 300 can include a radiant heater 350, which is shown as radiant heater 350 in FIG. 7. The heating of the inflatable float tank 300 can be achieved by installing the electric heater 350 over the interior floor of the tank 310 or upon a base 310 but under an internal liner of the inflatable float tank 300. These heater(s) 350 can be controlled with a thermostat control 387 to regulate the temperature between 88 and 99 degrees Fahrenheit, preferably between 91 and 95 degree Fahrenheit. An external heating system can also be implemented where the water is pumped by circulation pump 370 from the inflated and filled float tank 300, travels through an external heating chamber, then is returned to the float tank 300. This method of heating can be turned off for using the inflated and filled float tank 300 for long periods of time, however, thus the radiant heating exhibits some advantages in some embodiments of the invention as the float solution begins to cool without continuous heat. However, due to the increased insulation provided by the dual-wall drop-stitch design the heat retained may provide for extended periods without heating or circulation of the float solution.

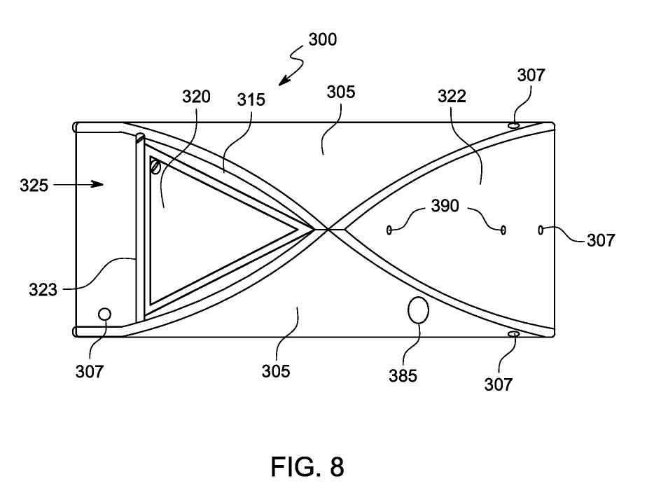

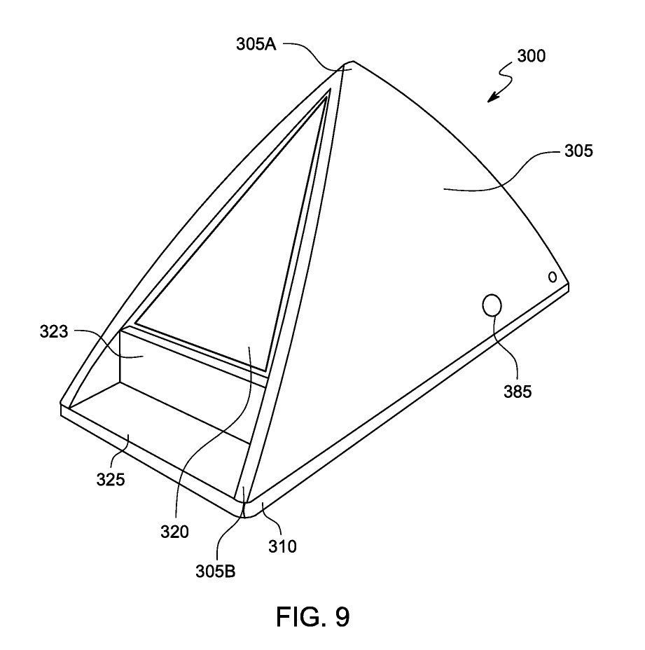

FIGS. 8 and 9 show a top view and perspective view respectively of the inflatable float tank 300, which has a rectangular bottom profile. The design of the float tank 300 may be described as a substantially pyramid-like design. The end walls 315 and 322 can be at least partially triangular in shape. The side walls 305 are sloped and triangular or pyramid shaped as shown. The side walls 305 may be described as being curved, non-flat, and devoid of a planar surface so as to resist condensation on an interior surface and dripping therefrom. As previously discussed the side walls 305 and end walls 315 and 322 as well as the door 320 can be vertically angled so as to inhibit dripping therefrom. The sides 305 and ends 315 and 322 may include one or more vent holes 390 for air circulation from the outside ambient air to the interior air within the float tank 300. The side walls 305 and end walls 315 and 322 may also have one or more air ports 307 for inflating the sections of the float tank 300, and one or more fluid ports 385 for accessing the float solution for circulation, filtration, and/or heating.

FIGS. 10A, 10B, 10C, 10D, 10E, and 10F illustrate assembly of the float tank. In FIG. 10A, a float tank is shown in a substantially deflated, collapsed and folded state. The state shown in FIG. 10A may be a float tank state for shipping, storing, or transporting the inflatable float tank. The float tank shown in FIG. 10A may be part of a kit include the float tank and may include a pump as shown in FIG. 10B, a bag of Epsom salt (not shown), a radiant heater, a circulation pump, and/or any other devices and components discussed herein or as seen fit to include along with the inflatable float tank.

FIG. 10B show the inflatable float tank being unfolded and laid flat by two people. Of course a single person or more than two people may perform such acts. FIG. 10C shows connection of an inflation pump to a port or inflation connection of the float tank. FIG. 10D shows the float tank partially inflated. FIG. 10E shows the float tank fully inflated and the upper portion being situated upon an inflated base portion. FIG. 10F shows the inflated float tank having been filled with heated highly salinated water and a person entering the inflated float tank through an inflated door and opening of the inflated float tank.

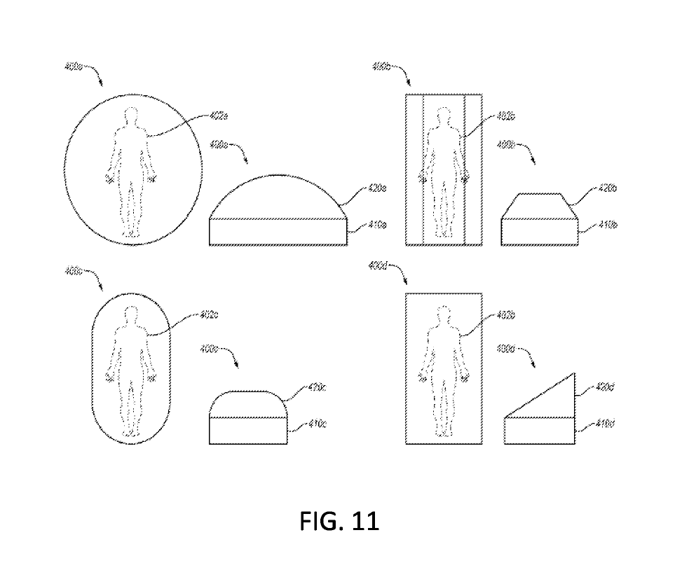

FIG. 11 illustrates different shapes of the inflatable float tank 400a, 400b, 400c, and 400d, which are not limiting because other shapes may be used. The parameters should allow for a subject (e.g., 402a, 402b, 402c, 402d) to float while relaxed without touching the perimeter edges. For example: the inflatable float tank 400a can have a circular-shaped inflatable portion 410a for a subject 402a to float in with an inflatable domed portion 420a; the inflatable float tank 400b can have quadrilateral-shaped inflatable portion 410b for a subject 402b to float in with an inflatable portion 420b having sloped ends and sides with a horizontal top; the inflatable float tank 400c can have an oblong-shaped inflatable portion 410c for a subject 402c to float in with a semi-domed inflatable portion 420c; or the inflatable float tank 400d can have a quadrilateral-shaped inflatable portion 410d for a subject 402d to float in with a triangle shaped inflatable portion 420d that has at least one inflatable wall. The vertical portions may be omitted and the inflatable portions 420a, 420b, 420c, and 420d may be the shape of certain embodiments. However, other shapes and configurations can be used. Such embodiments are devoid of rigid, metallic, and hard plastic structural supports.

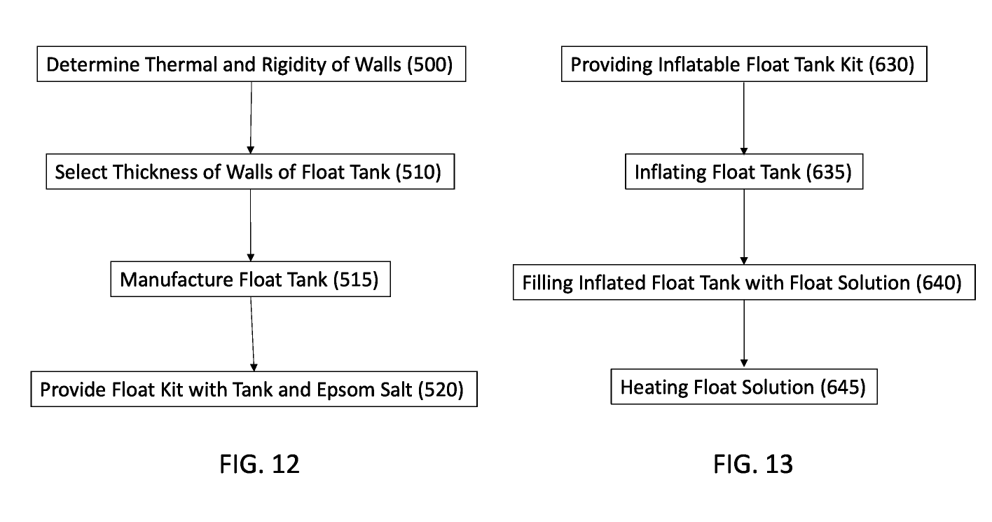

FIG. 12 illustrates a method for manufacturing and assembling an inflatable float tank as well as providing a kit for deployment of a float tank. The method includes determining a desired thermal attribute of the float tank and desired structural rigidity and strength attribute. (500). The thermal attribute of the walls and/or base can include an insulation rating or rate of heat transfer there through. The inflatable float tank having walls devoid of a collapsible internal support structure. The walls of the float tank can have a smooth fabric exposed to the subject. The smooth fabric can include a PVC coated polyester. The rigidity of the walls can include a lack of bending resistance when inflated so as to support at least 100 pounds without rigid supports.

The method of manufacture of the inflatable float tank (500) can include a drop stitch technique of manufacturing the inflatable walls. The drop stitch fabric can include opposing side walls having threads extending there between creating the insulation and structural support thereto. The drop stitch material of the walls, floor and/or door can include a V-stitch pattern where the drop stitch material includes internal tensile threads that are substantially inelastic so as to hold the walls at a set distance relative to one another yet provide the rigidity between the opposing walls due to the acute vector forces provided there between.

The method includes selection of the thickness of the walls for the float tank based on the desired attributes discussed herein and (510). The method considers the internal pressure inside the inflated walls, which is greater than the atmospheric pressure. As such, the walls become substantially rigid due to the internal support structure of the walls. The fluid for inflating the walls can be air and can be filled to a pressure of at least 15 psi. The air within the walls can also be inflated to a pressure of at least 30 psi. The walls can be comprised of a closed surface of flexible fabric able to be filled with air, making a rigid, but compliant structure that includes a pool portion. The float tank is manufactured from the drop stitchtitch material (515) and is sufficiently rigid and strong for supporting the floating solution when inflated. The floating solution including water and Epsom salt and is heated to a sufficient temperature between 88 and 99 degrees Fahrenheit, for example.

The inflatable float tank can be provided along with a kit (520) including Epsom salt and an air compressor for filling the inflatable float tank to a pressure of at least 15 psi, or greater than 30 psi. The inflatable float tank can be provided along with an electrical radiant heater configured to heat water in the pool and maintain the temperature of the water between 88 and 99 degrees Fahrenheit. The inflatable pool portion having walls and being dimensioned to hold less than 14 inches deep of water between a base and a surface of the water. In one embodiment, the floatation solution is less than 12 or 10 inches in depth. The base being defined by the walls and dimensioned sufficient for a subject to float in the water of the pool without touching the pool sides or the base of the pool.

The method of manufacturing the inflatable float tank includes providing a water circulation pump and a filter located within the float tank. The method of manufacturing the inflatable float tank can alternatively include providing a water circulation pump and a filter located outside of the float tank but in fluidic circulation via a conduit with the float solution provided within the float tank when inflated and filled with the float solution.

According to certain embodiments disclosed herein, the float tank base is rectangular shaped. The method can also include manufacturing an inflatable sidewall structure where the walls are triangular shaped with a base dimensioned so that each wall meets at an angle with respect to one another. The sides of the float tank for holding the float solution may be less than 28 inches high when inflated.

Referring to FIGS. 10A-10F and FIG. 13, a method for assembling an inflatable therapeutic float tank or float tank kit is illustrated. The therapeutic float tank provides a sensory deprivation state for a user in order to inhibit stimulus to the user while floating in the float tank when assembled. The method includes providing the inflatable float tank kit (630). The float tank kit includes a collapsed inflatable and water proof pool portion designed to be inflated to hold water that has at least 800 pounds of Epsom salt dissolved in the water. The kit further comprising a collapsed opaque inflatable canopy portion made of a material that inhibits light from entering the interior of the float tank when inflated. The inflatable float tank defining a shape of the side, front, and rear walls upon inflation. The walls being substantially rigid when inflated due to an internally manufactured support structure within the walls. The internally manufactured support structure within the walls can include a drop stitch support structure such as a V-drop stitch structure as disclosed herein. At least one of the walls having a closable opening for entering the therapeutic float tank when inflated.

The method of assembling the float tank kit can be performed indoors in a float room that can include inflating the float tank to define the shape of the inflated float tank (635). The walls of the float tank can be manufactured from an integrated sidewall in fluidic gaseous communication so as to inflate multiple sidewalls in a single inflation step. Upon inflation, the wall become substantially rigid under at least 15 psi, or at least 30 psi of air pressure due to a drop stitch construction of the walls of the float tank.

The method can further include filling the float tank to less than 14 inches, or less than 12 inches, of water that has at least 3.5% salinity (640). The method can further include heating the water to between 88 and 99 degrees Fahrenheit (645). The method can further include setting an electrical radiant heater to silently maintain the water at a temperature between 88 and 99 degrees Fahrenheit. A method of dis-assembling the inflatable therapeutic float tank kit can include removing the float solution of at least 3.5% Epsom salt dissolved in water from the inflated pool portion. The method of dis-assembling the inflatable float tank can further include deflating the sides and base of the float tank as well as the door. The inflation of the float tank defining the shape of the pool portion and canopy portion in a substantially rigid form when inflated. The float tank becoming substantially flexible when deflated and foldable as compared to the inflated state.

In one aspect, the inflatable float tank can include vents, which can be pipes passing through the walls of the tank (e.g., PVC pipe) that includes an elbow or turn to inhibit light from entering into the tank. In one aspect, the pump can be configured with a filter, and may include a mechanical filter and a UV filter. This pump may also include ozone or other units for cleansing the water.

In one embodiment, the inflatable float tank can include a heater that can self-regulate temperature by including sufficient thermocouples and components and electronics to modulate the temperature and to obtain a desired temperature of the water in the tank. In one aspect, the water can be heated with unique under-tank heaters that heat the water to skin temperature so the user can't feel the difference between the water and air in the inflatable float tank. A custom heater with temperature control can hold the water to within +/−1 degree with an integrated thermometer or thermostat system. The heater can operate at or near skin temperature, releasing a subtle, gentle heat into the water when needed. This results in a stable water temperature to encourage a deeper, more peaceful float. Even during longer floats there is no overall heat loss as the heater instantly responds to the smallest temperature change. The heater can be configured to substantially eliminate electromagnetic radiation (EMF) from the float environment to improve floatation experiences.

In one embodiment, the pool base of the inflatable float tank can include a base surface having insulation, where the insulation can be coupled or affixed with the base surface or the insulation can be a separate member that is placed into or under the base member of the pool. The insulating member can isolate the water from the ground, and thereby reduce noise while insulating the base. This allows less heating to maintain the proper or desired temperature that is set with the heater. The insulating member can be a radiant heat mat.

In one embodiment, the water in the tank can have 800 pounds of Epsom salt dissolved into the water (e.g., the base of the float tank having a base about 4 feet by 8 feet with water depth of 10 inches) so when a subject is in the water, they float completely and effortlessly. However, other salts or other salt amounts can be used to facilitate floating. The water can be brine, which can range from about 3.5% or 5% (e.g., salinity). The water may be saturated with the salt at a temperature of about 95 degrees Fahrenheit to 105 degrees Fahrenheit. In one example, the water can have salt at from 250 to 500 parts per thousand (PPT), from 300 to 400 PPT, from 325 to 375 PPT, or about 350 PPT at operating temperature which is skin temperature. Skin temperature can usually range from 92° F. to 99° F. However, the water can be adjusted to between 91 and 99 degrees Fahrenheit preferably about 93.5 degrees Fahrenheit. The salt can be any salt or mixture of salts. The water may be supersaturated by first heating the water to a much higher temperature, saturating the water with salt, and then lowering the water to operating temperature. In any embodiment, the water has sufficient salt to facilitate effortless floatation of a human subject, which can be a child, teen, or adult, and which may range in weights. The amount of salt can drastically vary depending on a number of circumstances. Accordingly, an embodiment of the invention includes the inflatable float tank and a sufficient amount of salt to float the user. As such, 800 pounds of Epsom salt can be used as an example +/−10%, 20%, 30%, 40%, 50%, 60%, or more for a pool having 4 feet by 8 feet with water depth of 10 inches. These parameters can be used to calculate the salinity or saturation, which can be applied to other water volumes in accordance with the invention.

In one aspect, the salt can be added to the water to obtain at least about 60% saturation, at least about 70% saturation, at least about 75% saturation, at least about 80% saturation, and at least about 90% saturation. In one embodiment, the saturation can be about 99-100%.

In one aspect, the heater can heat the water to a temperature between about 91 degrees Fahrenheit to about 99 degrees Fahrenheit or there between. In one example, the water is set at about 93.5° F. plus or minus 2 or 4 degrees Fahrenheit and circulation pump is used to circulate the solution. The pump can circulate the water periodically or sporadically to keep the water clean when not in use. Such water pumps are well known. In one embodiment, the inflatable float tank can include a UV sterilization unit that uses UV light to sterilize or otherwise kill harmful microbes. Such UV units are well known. In one embodiment, the inflatable float tank can include an ozone unit that produces ozone and introduces the ozone in the water to kill harmful microbes. Such ozone units are well known. Optionally, the pump and/or UV unit and/or ozone unit can be combined into a single unit as is common with hot tubs. In one embodiment, the floatation unit can include a filter unit to filter the water to cleanse contaminants from the water. The filter unit can include a micron bag filter to keep the water clean. The filter unit can be associated with the pump or include in the pump unit so that the pump pumps water through the filter.

In one embodiment, the shape and design of the tank can be configured for enhancing or otherwise improving the user experience during use, such as in relaxation, meditation, or sensory deprivation. The angle of the tank walls can be designed to repel humidity and prevent condensation accumulation and distracting water drips from the ceiling of the tank that may otherwise interrupt a relaxing or peaceful float session. The tank can also be big enough that the user can lie down in a relaxed position without touching or hitting any side wall or ceiling in any direction. This tank walls can be lightproof or opaque so once the user is inside and the entrance is closed, the user can't see anything and almost pitch black or lightlessness can be achieved.

In one embodiment, the angle of the tank walls is designed to inhibit condensation dripping back into the pool. There are a wide range of angles that can inhibit condensation dripping. It should be recognized that different materials with different properties (e.g., hydrophobicity compared to hydrophilicity) or surface treatments (e.g., smooth compared to rough) will have different angles that inhibit condensation drip. The angles can be a minimum of 1 inch vertical distance for every horizontal foot; however, steeper wall angles may be better such as 2 inches vertical distance for every horizontal foot, 4 inches vertical distance for every horizontal foot, 6 inches vertical distance for every horizontal foot, 8 inches vertical distance for every horizontal foot, 10 inches vertical distance for every horizontal foot, 12 inches vertical distance for every horizontal foot, 14 inches vertical distance for every horizontal foot, or steeper. Examples of angles can range from at least about 4.75 degrees, 9.15 degrees, 18.42 degrees, 26.74 degrees, 30 degrees, 40 degrees, 45 degrees, 60 degrees, or any value or range there between.

In one embodiment, the inflatable float tank can include the following features: 24/7 filtration, including when not in use; extremely low interior condensation; under-body heater, which can maintain constant temperature during floatation; UV sterilization; air venting to bring fresh air into the inflatable float tank; multi-color LEDs, which can be configured to pause on select colors (e.g., mood colors) or for strobe effects, or starry sky simulation; music capability (e.g., plugin for music device or Bluetooth); all components removable; silent pump motor, with one or multiple jets for better water flow; and internal or external control system and session control.

In one embodiment, the inflatable float tank can include an interior that is selectively lit by one or more underwater LED lights in any color, which can be set or change, such as a rich, vibrant blue. The lighting can be configured or programmed to gently fade in and out, and which may be controlled at the touch of the interior switch to enhance the peace of the floatation environment. Mood and color-changing lighting may also be available in the ceiling, such as distributed across the roof (e.g., star patterns) and highlighting the inflatable float tank.

In one embodiment, a user can use the inflatable float tank for relaxation, meditation, sensory deprivation, or other floating use. For example, the use can be floating in about 10 inches of water saturated with over 800 pounds of Epsom salts, which can simulate a near-zero gravity environment. The water is heated to skin temperature, which dulls the sense of touch. After a while, the user may not even feel the water. This allows the user to feel weightless, such as floating through space. The inflatable float tank environment can allow for no distractions, just stillness. This allows the stresses and worries to fade away. After a float tank session, the user's physical body can feel light and energized. Also, any physical pain experienced prior to the inflatable float tank session may slowly fade as inner peace arises. For example, the inflatable float tank can be used for: diminished physical pain, including chronic pain; accelerated healing; spinal decompression; stress relief; mental relaxation; enhanced creativity and learning; pregnancy relief; help with depression and anxiety; strengthened immune system; assistance in a more restful sleep (e.g., for those with insomnia); increased energy; enhanced mental focus and problem solving; anger management; deep meditation; or the like. Also, the inflatable float tank can be used to help treat or relieve symptoms of: arthritis, back pain, premenstrual tension, postpartum depression, asthma, migraine headaches, multiple sclerosis, cardiovascular conditions, osteoporosis, synovitis, and fibromyalgia, among others.

During use of the inflatable float tank, the inflatable float tank environment can leave the user feeling disconnected and isolated from the normal world of perception. This experience can be very relaxing and therapeutic for the mind as it does not have to take in or monitor normal senses. It is a great environment to think with extra brain power. Also, it presents the perfect meditation environment with the lack of mental stimulus.

In one embodiment, the inflatable float tank can be configured for use in sensory deprivation floatation therapy. Sensory deprivation therapy utilizes floating as a method of attaining the deepest relaxation a user can experience. This type of sensory deprivation therapy can help to ease a number of medical conditions and symptoms. During such a use, the lights, sounds, or other distractions that may arise from the components can be turned off or excluded.

In one embodiment, a method of floatation can include: providing the inflatable float tank as described herein, the inflatable float tank having water in the pool that has at least about 3.5% salinity and at least about 95° F.; and floating a subject in the water of the pool without touching the pool base or pool walls. In one aspect, the method can include inhibiting light and/or sound from entering into the inflatable float tank. In one aspect, the method can include the subject floating without their arms or legs touching another part of their body. In one aspect, the method can include the user floating until having one or more of the following: diminished pain; accelerated healing; spinal decompression; stress relief; mental relaxation; enhanced creativity and learning; pregnancy relief; relief from depression and/or anxiety; strengthened immune system; attainment of a more restful sleep; increased energy; enhanced mental focus; improved anger management; deep meditation; or combinations thereof. In one aspect, the method can include the user floating until improving: arthritis, back pain, premenstrual tension, postpartum depression, asthma, migraine headaches, multiple sclerosis, cardiovascular conditions, osteoporosis, synovitis, fibromyalgia, or combinations thereof.

In one embodiment, an inflatable float tank kit can include un-inflated components of the inflatable float tank as described herein. Such an inflatable float tank kit can include: an inflatable pool base and inflatable sidewalls configured to hold at least 8 inches of water, but less than 14 inches of water and having a pool base dimension sufficient for a subject to lay in the pool without touching pool sides. The inflatable float tank is configured to be inflated into a substantially rigid float tank with a high level of insulation due to the drop-stitch materials. In one aspect, the inflatable float tank kit can include a heating unit. In one aspect, the inflatable float tank kit can include a water circulation pump and filter. In one aspect, the inflatable float tank kit can include one or more of: a salt reservoir, a UV unit; an ozone unit; a timer; a light device; or a sound device. In one aspect, the inflatable float tank kit can include an amount of salt sufficient to create water salinity in the pool base of at least 3.5% salinity. One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “ a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tents, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims. All references recited herein are incorporated herein by specific reference in their entirety.

We claim:

1. A therapeutic inflatable float tank providing a sensory deprivation state for a subject in order to inhibit light, temperature, and physical stimulus to the subject while floating in the therapeutic inflatable float tank, comprising:
opaque light inhibiting inflatable walls, the walls including an internal drop-stitch support structure that lends rigidity to the walls when inflated but is substantially collapsible, flexible, and foldable when uninflated;
a heater configured to maintain a temperature of a float solution between 88 and 99 degrees Fahrenheit so as to inhibit temperature stimulus to the user; and
a base connected to the inflatable walls so as to provide a water-tight connection between the base and walls, the inflatable sides and base dimensioned to hold less than 24 inches deep of float solution and dimensioned sufficient for a subject to float in the float solution without touching the pool sides or the base of the pool.

2. The therapeutic inflatable float tank according to claim 1, the walls providing thermal insulation to the interior of the therapeutic inflatable float tank due to a substantially air-filled void between the drop-stitch connected inner surfaces of the walls.

3. The therapeutic inflatable float tank according to claim 1 being devoid of solid support structures.

4. The therapeutic inflatable float tank according to claim 1 being devoid of metal or plastic internal or external support structures.

5. The therapeutic inflatable float tank according to claim 1, the base being inflatable for providing thermal insulation between a floor and the interior of the inflatable float tank.

6. The therapeutic inflatable float tank according to claim 5, further comprising a radiant heater for substantially silent heating of the float solution during a float session.

7. The therapeutic inflatable float tank according to claim 5, the base having an internal drop-stitch support structure that lends rigidity to the base when inflated but is substantially collapsible, flexible, and foldable when uninflated.

8. The therapeutic inflatable float tank according to claim 1, the walls including an opening for the subject to enter the therapeutic inflatable float tank.

9. The therapeutic inflatable float tank according to claim 8, further comprising an inflatable pivotable door made having an internal drop-stitch support structure that lends rigidity to the door when inflated but is substantially flexible, collapsible, and foldable when uninflated.

10. The therapeutic inflatable float tank according to claim 1, at least two walls being seamless between opposing ends of the at least two walls.

11. The therapeutic inflatable float tank according to claim 1, at least two walls being triangular and curved in shape without planar surfaces so as to inhibit internal condensation drip therefrom.

12. The therapeutic inflatable float tank according to claim 1, at least two walls being devoid of seams and undulations.

13. The inflatable float tank of claim 1, wherein the walls are inflated to a pressure of at least 15 psi resulting in substantial rigidity of the walls as compared to the uninflated state of the float tank.

14. The inflatable float tank of claim 1, wherein the air within the walls is inflated to a pressure of at least 20 psi resulting in substantial rigidity of the walls.

15. The inflatable float tank of claim 1, further comprising the float solution including water having at least 3% salinity of Epsom salt dissolved therein.

16. The inflatable float tank of claim 1, further comprising the float solution including water having at least 800 pounds of Epsom salt dissolved in between 160 and 240 gallons of water held within the inflatable float tank resulting in at least 3% salinity of Epsom salt in the water.

17. A method for assembling the inflatable float tank according to claim 1, comprising:
inflating the inflatable float tank to a rigid upright position;
filling the inflatable float tank with between 8 and 14 inches of the float solution; and
heating the float solution to between 91 and 99 degrees Fahrenheit so as to provide a sensory deprivation float therapy session.

18. A kit for floatation therapy, comprising:
the inflatable float tank according to claim 1;
a heater;
a circulation pump;
a filtration system; and
800 pounds of Epsom salt.

19. The kit according to claim 18, wherein the kit is devoid of poles and rigid supports.

20. A method for manufacturing an inflatable float tank, comprising:
selecting a wall thickness for side walls of the inflatable float tank, the wall thickness being selected to provide thermal insulation and sidewall rigidity when inflated; and
manufacturing the inflatable float tank comprising a drop-stitch material, the drop stitch material includes opposing side walls with stitched threads extending at acute angles from one another, where when uninflated these internal stitch structures are allowed to collapse and provide a substantially foldable and pliable deflated float tank as opposed to substantially rigid sidewalls upon inflation.

21. A method for manufacturing an inflatable float tank according to claim 20, wherein at least two sidewalls are seamless from end to end.

22. A method for manufacturing an inflatable float tank according to claim 20, wherein the sidewalls are curved and devoid of planar surfaces.

23. A method for manufacturing an inflatable float tank according to claim 20, wherein the surface of the sidewalls are substantially continuous from a top of the sidewalls to a bottom of the sidewalls without undulations or seams.

24. A method for manufacturing an inflatable float tank according to claim 20, wherein the inflatable float tank is supported only by the internal stitch structure upon inflation.

25. An inflatable float tank, comprising:
opaque inflatable sidewalls including a drop-stitch material, the drop stitch material includes opposing walls with stitched threads extending at acute angles from one another, where when uninflated these internal stitch structures are allowed to collapse and provide substantially foldable and pliable deflated sidewalls as opposed to substantially rigid sidewalls upon inflation, the inflatable sidewalls being curved and devoid of seams and devoid of uneven surfaces so as to inhibit condensation dripping therefrom;
an inflatable base including a drop-stitch material, the drop stitch material includes opposing walls of the base with internal stitched threads extending at acute angles from one another, where when uninflated these internal stitch structures are allowed to collapse and provide a substantially foldable and pliable deflated base as opposed to the substantially rigid base upon inflation;
an opening in a wall for a subject to enter the interior of the inflatable float tank;
between 160 and 280 gallons of float solution, the float solution comprising water and at least 700 pounds of Epsom salt dissolved in water so as to provide a buoyant float condition; and
a heater for heating the float solution to between 91 and 95 degrees Fahrenheit so as to inhibit sensory stimulation during the float session.
